(19) United States
(12) Patent Application Publication
(10) Pub. No.: US 2022/0395204 A1
(43) Pub. Date: Dec. 15, 2022

(54) DEVICE FOR WITHDRAWAL AND PROCESSING OF A SAMPLE

(71) Applicants: UNIVERSITÉ CATHOLIQUE DE LOUVAIN, Louvain-la-Neuve (BE); MEDI-LINE SA, Angleur (BE)

(72) Inventors: Jean-Luc BALLIGAND, Kraainem (BE); Irina LOBYSHEVA, Bruxelles (BE); Nancy VAN OVERSTRAETEN, Dongelberg (BE); Jonathan MAIRE, Rocourt (BE); Jean MEENS, Liège (BE); Patrick MERKEN, Houtain-Saint-Siméon (BE)

(73) Assignees: UNIVERSITÉ CATHOLIQUE DE LOUVAIN, Louvain-la-Neuve (BE); MEDI-LINE SA, Angleur (BE)

(21) Appl. No.: 17/777,101

(22) PCT Filed: Nov. 21, 2020
(86) PCT No.: PCT/EP2020/082996

§ 371 Date: May 16, 2022

(30) Foreign Application Priority Data
Nov. 22, 2019 (EP) 19210950.2

(51) Int. Cl. A61B 5/15

(52) U.S. Cl. CPC A61B 5/150236; A61B 5/15003; A61B 5/150244; A61B 5/150755; A61B 5/150763; A61B 5/150916

(57) ABSTRACT

A device (100) having a proximal end (20) and end a distal end (40) for withdrawal of a liquid sample, fractionation, and separation of one or more fractions comprising: a piston assembly (300) comprising a piston (302) dismountably attached at its proximal end (20) to an actuating rod (350); a container (200) having a body (202) disposed with a cylindrical chamber (204) for slidable movement of the piston (302) therein; wherein the cylindrical chamber (204) is provided at the distal end (40) with a resealable septum (410) for co-operation with a puncture needle assembly (600) for withdrawal of the liquid sample, an at the proximal end (20) with a stop member (206), which stop member (206) limits movement of the piston (300) in a proximal direction, wherein the container body (202) is dimensioned to fit inside a centrifuge rotor, and is configured for breakability into two parts at a temperature of 0 deg C. or lower at a breakable zone (210).

### FIELD OF THE INVENTION

The invention is in a field of withdrawing and processing a liquid sample, in particular a device for same, in particular for a blood sample.

### BACKGROUND TO THE INVENTION

Accurate assaying of the level of markers in samples, such as the bioavailable nitric oxide (NO) in vivo in human circulation, is important for the protection of human health.

In the case of NO, insufficiency of NO production from the endothelium is a crucial sign of endothelial dysfunction in many metabolic diseases and, especially, cardiovascular diseases developed under various risk factors including age, hypertension, smoking, and hypercholesterolemia. The functionality of the endothelial nitric oxide synthase (eNOS) and NO bioavailability in the vascular bed in vivo are difficult to assess quantitatively, especially in humans.

Blood levels of heme(Fell)-nitrosyl complexes (HbNO) are used for assay the vascular NO availability, but the time taken to process a blood sample is of crucial importance in order to conserve the initial HbNO levels. There is a requirement to draw a sample rapidly from the subject, transport and process the sample with a minimum of steps to avoid blood oxygenation, and/or HbNO complex degradation.

The prior art describes several devices for extraction of blood but none solve the problem of separation of a fractioned sample requiring additional equipment and delays in processing leading to sample deterioration. U.S. Pat. No. 5,174,301 discloses a syringe with a removable piston rod wherein fractions are extracted using a separate processing step and device. U.S. Pat. No. 5,174,301 discloses a syringe with a one-way no return piston; the device does not allow integrated sample processing. U.S. Pat. No. 3,577,980 discloses a syringe with a removable piston rod; the device does not allow integrated sample processing.

The present invention provides a device aimed at overcoming the problems of the art.

### SUMMARY OF THE INVENTION

The invention pertains to a container with a piston connected to a removable rod that allows the collection of a fluid, such as blood, its centrifugation and subsequent freezing, all without exposure of the collected fluid to ambient air. The centrifugation fractionates the fluid.

Described herein is a device (100) having a proximal end (20) and a distal end (40) for withdrawal, processing and storage of a liquid sample comprising:
a piston assembly (300) comprising a piston (302) dismountably attached at its proximal end (20) to an actuating rod (350),
a container (200) having a body (202) disposed with a cylindrical chamber (204) for slidable movement of the piston (302) therein, wherein the cylindrical chamber (204) is provided:
at the distal end (40) with a resealable septum (410) for co-operation with a puncture needle assembly (600) for withdrawal of the liquid sample, and
at the proximal end (20) with a stop member (206), which stop member (206) limits movement of the piston (300) in a proximal direction.

The body (202) may be dimensioned to fit inside a centrifuge rotor.

Also described herein is a device (100) having a proximal end (20) and a distal end (40) for withdrawal of a liquid sample, fractionation, and separation of one or more fractions comprising:
a piston assembly (300) comprising a piston (302) dismountably attached at its proximal end (20) to an actuating rod (350),
a container (200) having a body (202) disposed with a cylindrical chamber (204) for slidable movement of the piston (302) therein, wherein the cylindrical chamber (204) is provided:
   at the distal end (40) with a resealable septum (410) for co-operation with a puncture needle assembly (600) for withdrawal of the liquid sample, and
   at the proximal end (20) with a stop member (206), which stop member (206) limits movement of the piston (300) in a proximal direction,
      wherein the container body (202) is configured as centrifugation tube, and is configured for breakability into two parts at a temperature of 0 deg C. or lower at a breakable zone (210).

The actuating rod (350) may be configured for non-returnable movement in a proximal (20) direction.

The device (100) may be provided with a linear ratchet mechanism for non-returnable movement of the actuating rod (350) in the proximal (20) direction.

The piston (302) and actuating rod (350) may be configured such that the actuating rod (350) is dismountable from the piston (300) by application of an axial pulling force by the actuating rod (350) against the piston (302) engaged in the stop member (206).

The container body (202) may be configured for breakability into two parts at a temperature of 0 deg C. or lower at a breakable zone (210) by the application of mechanical force.

The container body (202) may be weakened at the breakable zone (210).

The piston (302) may be dismountably attached to the actuating rod (350) by a latching joint, wherein the latching joint comprises:
a first part (320) on the piston (302) comprising one or more rounded protrusions; and
a second part (352) on the actuating rod (350) comprising one or more recesses or holes for retaining the rounded protrusion.

The container body (202) at the proximal end (20) may be bevelled, tapered, conical or rounded for insertion into and/or stabilisation in a centrifuge rotor.

The container body (202) may be configured for centrifugation at at least 1000 g for at least 10 minutes.

The device (100) may be configured for to withstand freezing at a temperature of up to −200 deg C.

The cylindrical chamber (204) may be disposed with a composition comprising one or more substances having an antioxidant property and/or one or more substances having an anticoagulant property.

The distal end (40) of the body may be configured to engage with a holder (500) disposed with a cylindrical passage (508) open at a proximal end (20) dimensioned to receive the distal end of the device (100), wherein the distal end (40) of the passage (508) terminates in a fitting (506) for connection to the puncture needle assembly (600).

The device (100) may further comprise an identifier, such as writable label, a writable space, a programmable or non-programmable RFID tag, an optically-readable 1- or 2-dimensional code.

Further provided is a use of a device (100) as described herein, for separation of erythrocytes from a blood sample, wherein after centrifugation of the blood sample in the container (200), the centrifuged sample in the container (200) is frozen, the container body (202) broken at the breakable zone (210), and the erythrocytes are retained in the proximal part of the cylindrical chamber (204).

Further provided is a method for fractionating, and separating one or more fractions from a blood sample comprising:
obtaining a device (100) according to any of claims 1 to 12 containing the blood sample,
fractionating the sample by centrifugation,
freezing the device (100) after fractionation, and
breaking the container body (202) at the breakable zone (210),
   wherein the one or more fractions are separated in one part of the container body (202) broken at the breakable zone (210).

### FIGURE LEGENDS

FIG. 1 is a cross-sectional view of a container part of the device described herein.

FIG. 2 is a cross-sectional view of a piston assembly part of the device described herein.

FIG. 3 is an isometric and exploded view of the device described herein.

FIG. 4 panels A to D shows cross-sectional vie of a status of the device at different steps of sample withdrawal.

FIG. 5 is a cross-sectional view of the container part of FIG. 1, wherein a breakable zone is indicated.

FIG. 6 is a cross-sectional view of a piston and dismountable coupling.

FIG. 7 is a cross-sectional view of an actuating rod and dismountable coupling.

FIG. 8 is a cross-sectional view of a container part of the device described herein, wherein the resealable septum is disposed in an end cap.

FIG. 9 is a cross-sectional view of the end cap of FIG. 8.

FIG. 10 is a cross-sectional view of a holder.

FIG. 11 is a cross-sectional view of a puncture needle assembly.

FIG. 12 is a cross-sectional view of a puncture needle assembly mounted in the holder.

FIG. 13 is a cross-sectional view of a holder and puncture needle assembly engaged in the device and puncturing the seal.

### DETAILED DESCRIPTION OF INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. It is to be understood that other embodiments may be utilised and structural or logical changes may be made without departing from the scope of the present invention. The following detailed description, therefore, is not to be taken in a limiting sense, and the scope of the present invention is defined by the appended claims.

The terms “distal”, “distal end”, “proximal” and “proximal end” are used through the specification, and are terms generally understood in the field to mean towards (proximal) or away (distal) from the surgeon side of the apparatus. Thus, “proximal (end)” means towards the practitioner side and, therefore, away from the patient side. Conversely, “distal (end)” means towards the patient side and, therefore, away from the practitioner side.

Presently described is a device having a proximal end and a distal end for withdrawal, processing and storage of a liquid sample. In particular the device is for withdrawal of the liquid sample, (optionally storage), fractionating, and separation of the fractions from the sample. The device comprises a container having a body disposed with a cylindrical chamber for slidable (axial) movement of a piston therein. The device comprises further comprises a piston assembly comprising a piston dismountably attached at its proximal end to an actuating rod. Movement of the piston in the proximal direction creates suction at the distal end of the cylindrical chamber for drawing up the liquid sample therein.

Provided at the proximal end of the cylindrical chamber is a stop member. The stop member engages with the proximal end of the piston. The stop member limits proximal movement of the piston. In other words, the stop member limits axial movement of the piston in the proximal direction. The piston engaged with stop member may further (hermetically) seal the proximal end of the cylindrical chamber.

The piston and actuating rod are configured such that the actuating rod is dismounted from the piston by application of pulling force against the piston engaged in the seating.

The device is capable of retaining a liquid sample in the cylindrical chamber under tight conditions. It may withstand freezing at a temperature of up to −200 deg C. It is sterilisable e.g. by radiation (e.g. gamma, X-Ray or E-Beam depending on the resistance of composition in the cylindrical chamber to radiation). It is centrifugable at speeds at least of 1000 g for 10 minutes. Centrifugation fractionates the liquid sample. It can hold a composition in the cylindrical chamber for reaction with the withdrawn liquid sample. The device may be a one-time use device.

The device may be disposed with an identifier such as writable (by pen, marker) label, a writable space, a programmable or non-programmable RFID tag, 1- or 2-dimensional optically-readable code (e.g. a barcode, QR code).

The device allows collection/centrifugation (for isolation of plasma and erythrocyte fractions), fast freezing of these fractions for long-time conservation without extra-exposure of blood to oxygenation/oxidation, and following mechanical separation of the fractions for any potential analysis. The potential analysis may be, for instance, the HbNO assay in the erythrocytes (when antioxidant/anticoagulant added); or NOx, surrogate NO biomarker (nitrite/nitrate as NO adducts availability) in erythrocytes and in plasma; any biomarker. Erythrocytes/plasma samples can be potentially used for analysis of tracing components by mass-spectrometry.

The piston is slidably mounted in the cylindrical chamber. The piston comprises a cylindrical sealing part that forms an annular seal against the inner wall of the cylindrical chamber. The seal allows the cylindrical chamber to develop a vacuum when the piston is withdrawn in the proximal direction. The cylindrical sealing part may be made from low-density polyethylene (LDPE).

The piston further comprises a dismountable coupling for dismountable attachment of the actuating rod. The dismountable coupling may be disposed at a proximal side of the cylindrical sealing part. The dismountable coupling may comprise one part of a latching joint. A latching joint has a first part and a second part, mutually dismountable, where both parts latch or temporarily connect to form a stable attachment that is dismounted by an application of a sliding force to one or both parts. In an exemplary latching joint, a first part comprises a rounded protrusion that may be, for instance, dome or hemi-spherical shaped, and a second part comprises one or more recesses or holes for retaining the rounded protrusion. Both parts are maintained attached under force, for example, by a spring. By a slidable movement of the rounded protrusion or of the recesses or holes, the rounded protrusion exits the recess thereby freeing the respective first part and a second part. The other part of the latching joint is disposed on the actuating rod described later below.

The piston further comprises a stop member surface that engages with a stop member disposed in the cylindrical chamber. The piston stop member surface engages with cylindrical chamber stop member to limit proximal movement of the piston. The piston stop member surface engaging with the cylindrical chamber stop member may further (hermetically) seal the proximal end of the cylindrical chamber.

An exemplary piston stop member surface has a shape of an annular ring that faces the proximal direction. A central part of the annular ring may be provided with the dismountable coupling. The piston stop member surface annular ring engages with a complementary annular surface of a stop member within the cylindrical chamber.

The cylindrical sealing part of the piston may have a maximum diameter of 4-5 mm, preferably of 4.5 mm. The dismountable coupling part of the piston may have a maximum width of 6-7 mm, preferably of 6.8 mm. It may be made from any suitable material for a piston that can withstand normal laboratory sample processing activities, including freezing at temperature up to −200 deg C., centrifugation at least 1000 g for 10 minutes, storage. It may be made from low-density polyethylene (LDPE).

The actuating rod, dismountably attached to the piston, is used to withdraw the piston in a proximal direction for uptake of liquid in the cylindrical chamber. When the piston reaches the limit of its movement, it engages with the stop member, and the continued application of a pulling force causes the actuating rod to detach from the piston. The actuating rod may comprise a handle, preferably a pull handle, at the proximal end.

The actuating rod comprises a dismountable coupling for dismountable attachment of the actuating rod. The dismountable coupling may be disposed at a distal side of the actuating rod. The dismountable coupling may comprise one part of a latching joint. As already mentioned elsewhere herein, a latching joint has a first part and a second part, mutually dismountable, where both parts latch to form a stable attachment that is dismounted by an application of a sliding force to one or both parts. A second part may comprise one or more recesses for retaining the rounded protrusion. Both parts are maintained attached under force, for example, by a spring. By a slidable movement of the rounded protrusion or of the recesses, the rounded protrusion exits the recess thereby freeing the respecting first part and a second part. The other part of the latching joint is disposed on the piston described elsewhere herein. The actuating rod may be removed prior to freezing and/or centrifugation.

The actuating rod may be configured for sliding only in one direction. It may be configured for sliding in the proximal direction only. It may be configured for non-returnable movement in the proximal direction. One-way sliding prevents liquid in the cylindrical chamber from returning to the subject. One-way sliding may be implemented by providing a linear ratchet mechanism. A linear ratchet may be disposed on the actuating rod, and a pawl provided at a proximal end of the container, on the body of the container. The linear ratchet engages with the pawl, thereby preventing movement of the actuating rod in a distal direction.

A maximum length of the actuating rod may be 107-108 mm, preferably 107.4 mm.

It may be made from any suitable material for an actuating that can withstand normal laboratory sample processing activities. It may be made from rigid ABS (Acrylonitrile butadiene styrene).

The container has a body disposed with a cylindrical chamber for slidable movement of a piston therein. The container holds liquid drawn into the cylindrical chamber.

The wall of the cylindrical chamber is rigid, so as to create a vacuum while the piston is withdrawn. A maximum diameter of cylindrical passage may be 4-5 mm, preferably 4.5 mm. A maximum length of the cylindrical chamber may be 105-115 mm, preferably 110 mm. The cylindrical chamber may have a maximum liquid holding volume of 1.3-2.3 ml, preferably of 1.75 ml.

A proximal end of the cylindrical chamber is provided with a stop member that engages with the body of the piston, namely with the piston stop member surface. The stop member is disposed at a proximal end of the cylindrical chamber. An exemplary stop member surface has a shape of an annular ring. A central part of the annular ring may be dimensioned to receive by insertion the dismountable coupling of the piston. The stop member engages with a complementary stop member surface of the piston.

The distal end of the cylindrical chamber may be sealed by a resealable septum. A resealable septum is an hermetic seal that is puncturable by a puncture needle. Removal of the puncture needle reseals the septum. It is appreciated that the puncture needle is provided with a lumen for the passage of the liquid sample. The resealable septum closes the distal end of the cylindrical chamber. The resealable septum may be disposed directly across the distal end of the cylindrical chamber. The resealable septum may be provided in an end cap mounted on the distal end of the cylindrical chamber.

The resealable septum may be made from any suitable material for a piston that can withstand normal laboratory sample processing activities, including freezing at temperature up to −200 deg C., centrifugation at least 1000 g for 10 minutes, storage. It may be made from butyl rubber.

The container body may be configured as a centrifugation tube. The exterior of the container body may be dimensioned to fit inside a centrifuge rotor. The container body may be configured for centrifugation at least at 1000 g for at least 10 minutes.

The container body at the proximal end may be bevelled, tapered, conical, preferably at an angle of 28 to 32 deg, for insertion (proximal end first) into a centrifuge rotor. The proximal end may be truncated. The container body at the proximal end may be rounded.

The exterior of the container body may take a form of a Falcon centrifuge tube.

The maximum outer diameter of container body may be 16-17 mm, preferably 16.5 mm.

The maximum length of container body may be 105-115 mm, preferably 110 mm.

The container body may be made from any suitable material for a container that can withstand normal laboratory sample processing activities, including freezing at temperature up to −200 deg C., centrifugation at least 1000 g for 10 minutes, storage. It may be made from polypropylene.

The cylindrical chamber may be supplied with a composition for mixing with the withdrawn liquid sample entering into the cylindrical chamber. The composition may be provided in a powdered form. The composition may comprise one or more substances having an antioxidant property. The composition may comprise a composition comprising one or more substances having an antioxidant property and/or one or more substances having an anticoagulant property. It is appreciated that a substance having an anticoagulant property may or may not also have an antioxidant property. It is appreciated that a substance having an antioxidant property may or may not also have an anticoagulant property. The substance having an antioxidant property may be selected from the group of N-acetyl cysteine, alpha-tocopherol, Trolox, ascorbic acid. The composition may comprise mixtures thereof such as a mixture of alpha-tocopherol and ascorbic acid, a mixture of alpha-tocopherol and N-acetyl cysteine. The substance having an anticoagulant property may be ethylenediaminetetraacetic acid (EDTA), or heparin. Preferably the composition comprises a mixture of heparin, Ascorbic acid and N-Acetyl-Cysteine. The composition may be provided in a gel, powdered, granular, or tablet form. The composition may additionally or alternatively comprise one or more tracing compounds.

The composition may contain a Ruthenium(II) complex-based sensor, for instance ([Ru(bpy)₂(dabpy)]²⁺) luminescence probe (A Novel Ruthenium-based Molecular Sensor to Detect Endothelial Nitric Oxide. Achini K. Vidanapathirana, Benjamin J. Pullen, Run Zhang, MyNgan Duong, Jarrad M. Goyne, Xiaozhou Zhang, Claudine S. Bonder, Andrew D. Abell, Christina A. Bursill, Stephen J. Nicholls & Peter J. Psaltis Scientific Reports volume 9, Article number: 1720 (2019). The probe detects nitric oxide in blood and plasma. This sensor has rate limiting steps, and is controlled by fast freezing after interaction with blood/plasma for post analysis.

The composition may contain an EPR spin probe of oxidative stress, such as cyclic hydroxylamine (Oxidative stress in healthy pregnancy and preeclampsia is linked to chronic inflammation, iron status and vascular function Dominique Mannaerts, Ellen Faes, Paul Cos, Jacob J Briedé, Wilfried Gyselaers, Jerome Cornette, Yury Gorbanev, Annemie Bogaerts, Marc Spaanderman, Emeline Van Craenenbroeck, Yves Jacquemyn PLoS One 2018 Sep. 11; 13(9):e0202919. doi: 10.1371/journal.pone.0202919. eCollection 2018). The probe may be used to assay of oxidative stress in blood.

The container body may be breakable at a low temperature (e.g. at a temperature at which the sample is frozen, or 0 deg C., or −18 deg C., or lower) at a predefined position, known as a breakable zone. The application of mechanical force causes the container body to break into two parts either side of the breakable zone at the low temperature. The breakable zone may be disposed with a weakened container body such as a notch or groove or thinner wall to facilitate breakage. The weakening of the body may have a continuous or discontinuous path e.g. a continuous or discontinuous ring. The position may be axial. The breakable zone may have a shape of an annular (ring). Breakage may be by application of a mechanical force, e.g. using a tool and/or manually (by hand). The mechanical force is a bending force.

The breakable zone facilitates breakage of the container body and its contents into two parts when the contents are frozen. When frozen, the container body at the breakable zone in particular is more brittle, and the container body and its frozen contents can be snapped into two parts. The breakability of the container body increases at a lower temperature. The breakability of the container body is enhanced at a temperature lower than ambient temperature, in particular at e.g. 0 deg C. or lower, or −18 deg C. or lower. The breakable zone may be disposed in the proximal half of the container body. The breakable zone may be disposed in the proximal third of the container body. Most preferably, the position of the breakable zone is at 30% of the total length of the container from the proximal end.

After centrifuging to fractionate the blood sample, for instance, to separate erythrocytes from a blood sample (e.g. 1000 g for 10 minutes), the container may be frozen (e.g. at −80 deg C.), and the container body subsequently broken into two parts at the breakable zone. The erythrocytes are retained in the proximal part of the cylindrical chamber. They may be recovered by advancing the piston in the distal direction; the piston may be advanced using, for instance, a rod without the linear ratchet thereby allowing movement of the rod in the distal direction and ejection of the frozen erythrocyte portion of the sample.

The sample may be any liquid sample, preferably a bodily fluid. Example of bodily fluid include blood, spinal fluid, interstitial fluid, peritoneal, and the like. Preferably the sample is blood.

The distal end of the container may be configured to engage with a holder. A holder comprises a body disposed with a passage open at a proximal end dimensioned to receive a distal end of the device. The distal end of the passage terminates in a fitting for connection to a puncture needle assembly. The puncture needle assembly comprises a puncture needle for puncture of the resealable septum that is connected directly or via a tube to a cannula for puncture of a vein or body cavity. The puncture needle assembly engaged in the fitting is configured to puncture the resealable septum. The fitting may be a Luer screw fitting. The puncture needle assembly may be BD Vacutainer® Multiple Sample Luer Adapter Ref 367300, or equivalent. The device may be provided with a (dismountable) puncture needle assembly holder (500)

The device may be provided with a holder comprising a body disposed with a cylindrical passage open at a proximal end dimensioned to receive a distal end of the device. The distal end of the passage terminates in a fitting for connection to the puncture needle assembly. The device may be provided with a (dismountable) holder with a puncture needle assembly.

The device described herein may be used in a method for fractionating and separating fractions from a sample. The actuating rod may be dismounted prior to freezing and/or centrifugation.

Provided herein is a use of a device described herein incorporating the breakable zone for separation of one or more fractions from a blood sample, wherein after centrifugation of the blood sample in the container, the centrifuged sample in the container is frozen, the container body broken at the breakable zone, and the one or more fractions are retained in one part (e.g. proximal or distal) of the cylindrical chamber broken at the breakable zone (210).

Also provided herein is a use of a device described herein incorporating the breakable zone for separation of erythrocytes from a blood sample, wherein after centrifugation of the blood sample in the container, the centrifuged sample in the container is frozen, the container body broken at the breakable zone, and the erythrocytes are retained in the proximal part of the cylindrical chamber.

Also provided herein is a method for fractionating and separating and one or more fractions from a blood sample comprising:
obtaining a device (100) as described herein (already) containing the blood sample,
fractionating the sample by centrifugation,
freezing the device (100) after fractionation, and
breaking the container body (202) at the breakable zone (210),
   wherein the one or more fractions are separated in one part (e.g. proximal part) of the container body (202) broken at the breakable zone (210).

Also provided herein is a method for fractionating and separating erythrocytes from a blood sample comprising:
obtaining a device (100) as described herein (already) containing the blood sample,
fractionating the sample by centrifugation,
freezing the device (100) after fractionation, and
breaking the container body (202) at the breakable zone (210),
   wherein the erythrocytes are contained in the proximal part (e.g. proximal part) of the container body (202) broken at the breakable zone (210).

The configuration provides a gastight cylindrical chamber that can hold a liquid sample for a period of time (e.g. 30-60 minutes) to allow freezing and/or an analysis, and without oxygenation. When the sample is blood, the centrifuge-compatible container allows separation of erythrocytes from the remainder. The device allows, for example, a measurement of nitrosylated haemoglobin in blood sometime after collection of the sample by the device with reduced oxidation. The breakable zone facilitates removal of erythrocytes from a blood sample as a frozen block which reduces sample oxygenation/oxidation caused by steps of liquid sample transfer (e.g. pipetting, pouring). The ability to freeze the sample and break the container body removes extra steps for fraction removal by pipetting, the need for additional equipment, and contamination-avoidance measures; centrifugation and separation are performed with the one device. As separation is performed under freezing conditions, there is less oxygenation which is critical for applications including assaying vascular NO. Contamination or mixing of fractions is avoided as the sample remains solid during separation.

### Examples

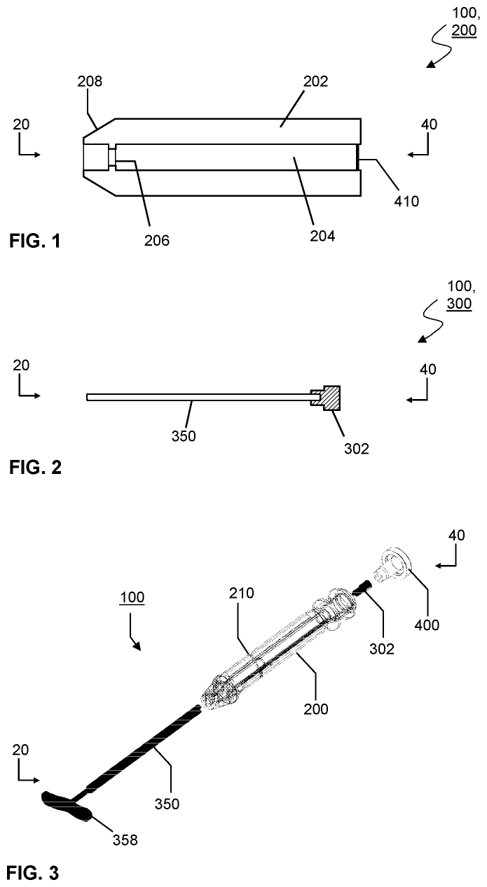

FIG. 1 is an example of a container (200) of a device (100) described herein having a proximal (20) and distal end (40). The container (200) has a body (202) provided with a cylindrical chamber (204). The proximal end (20) of the container (200) is provided with a stop member (206) configured to engage and limit proximal movement of a piston (300, FIG. 2). A resealable septum (410) is disposed over the distal end (40) of the cylindrical chamber (204). The proximal end (20) of the container body (200) may have a conical (208) outer surface.

FIG. 2 is an example of a piston assembly (300) of a device (100) described having a proximal end (20) and a distal end (40). The piston assembly (300) comprises a piston (302) that slidably engages in the cylindrical chamber (204). The piston assembly (300) further comprises an actuating rod (350) having a proximal end (20) and a distal end (40). The actuating rod (350) is dismountably attached to the proximal end (20) of the piston (302).

FIG. 3 is an isometric and exploded view of an example of a device (100) showing the piston (302), actuating rod (350), end cap (400), and container (200). The actuating rod (350) at the proximal end (20) is disposed with a pull handle (358). Further indicated is a breakable zone (210),

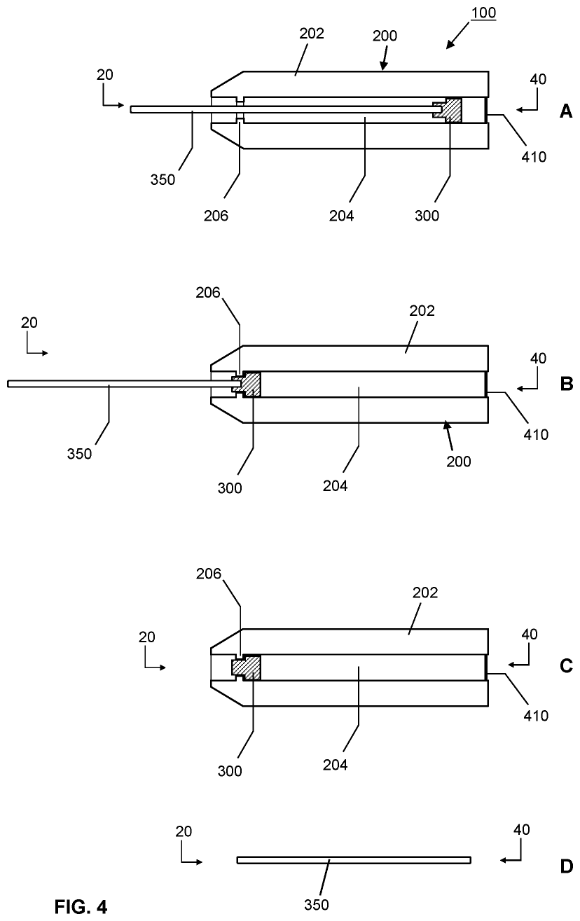

FIG. 4 panels A to D shows a sequence of steps for withdrawing a liquid sample. The piston (300) is positioned at a starting position a distal position within the cylindrical chamber (204) (A). The piston (300) is positioned at an end position that is the distal-most position within the cylindrical chamber (204) (B) by withdrawal of the actuating rod (350) in a proximal direction such that the piston contacts the stop member (206). The application of force in a proximal direction to the actuating rod (350) detaches the actuating rod (350) from the piston (300) (C, D).

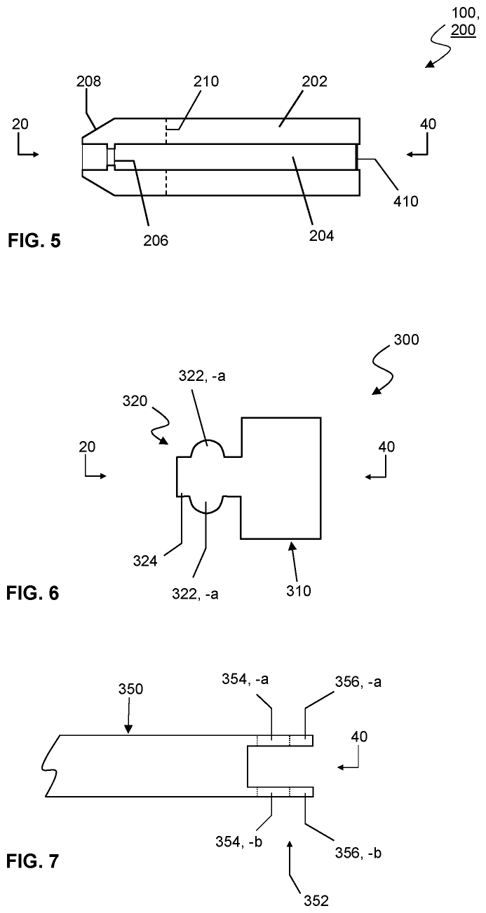

FIG. 5 is an example of a container (200) of a device (100) as shown in FIG. 1, wherein the container (200) has a body (202) provided with a breakable zone (210) disposed with a weakened part such as a notch or groove or thinned wall to facilitate breakage into two parts.

FIG. 6 Shows a cross-section of a piston (300) comprising a cylindrical sealing part (310) that forms an annular seal against the inner wall of the cylindrical chamber, and a dismountable coupling (320) for dismountable attachment of the actuating rod (350). The dismountable coupling comprises a first part of a latching joint that is a dome-shaped protrusion (322) provided on an elongated member (324) that extends in an axial and proximal (20) direction. The dome(s) protrudes radially from a central axis. In FIG. 6, two opposing domes (322, -a, -b) are provided on the elongated member (324).

FIG. 7 Shows a cross-section of a distal end (40) of an actuating rod (350), comprising a second part (352) of a latching joint comprising two arms (356, -a, -b) extending in an axial distal (40) direction. The arms (356, -a, -b) are configured to flank the elongated member (324) of the piston (300). Each arm (356, -a, -b) is provided with a circular hole (354, -a, -b) that are aligned in an axial direction. The pair of holes (354, -a, -b) engage with the two opposing domes (322, -a, -b), thereby latching the actuating rod (350) to the piston (300).

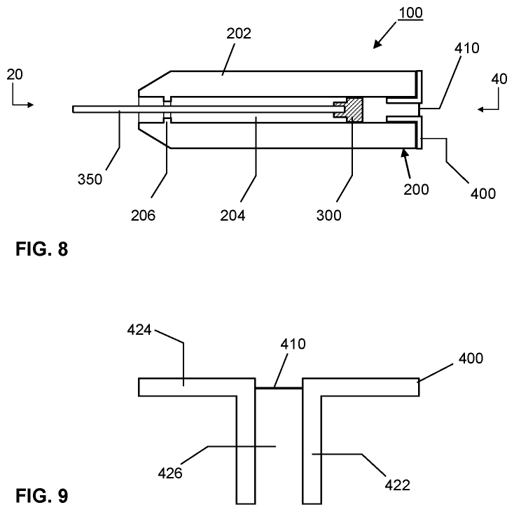

FIG. 8 illustrates a cross-section of the device (100) of FIG. 3 wherein the resealable septum (410) is provided in an end cap (400) that engages in the cylindrical chamber (204).

FIG. 9 illustrates a cross-section of the end-cap of FIG. 8. The end cap (400) is a flange having a hollow tubular part (422) at the proximal end, an annular plate like body (424) at the distal end, and a passage (426) passing therethrough. The resealable septum (410) closes passage (426).

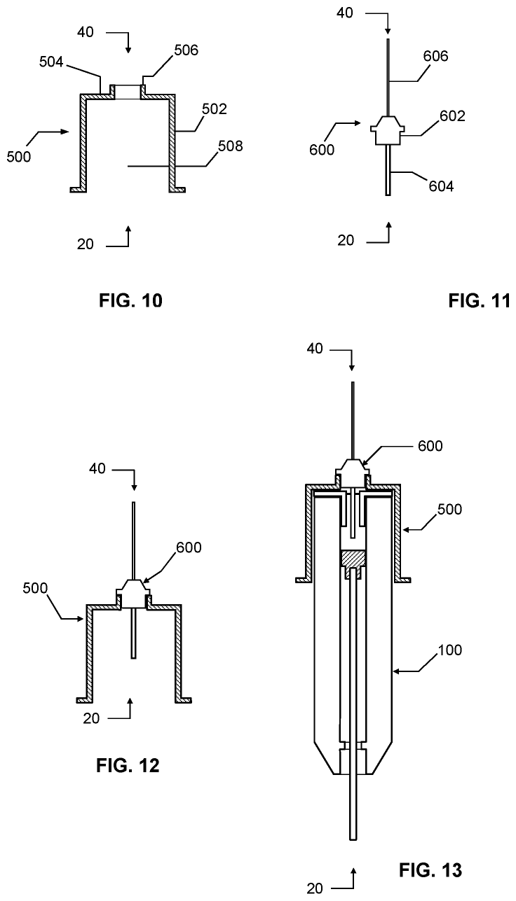

FIG. 10 illustrates a cross-section of a holder (500) comprising a body (502) disposed with a cylindrical passage (508) open at a proximal end (20) dimensioned to receive a distal end of the device (100). The distal end (40) of the passage (508) terminates in a fitting (506) for connection to a puncture needle assembly (600, see FIG. 11).

FIG. 11 illustrates a puncture needle assembly (600) comprising a complementary fitting (602) for attachment to the holder (500) fitting (506) that supports a puncture needle (604) projecting from a proximal side (20). The puncture needle (604) lumen is in fluid connection with a lumen of a cannula (606) projecting from a distal side (40) of the complementary fitting (602). The cannula (606) is for insertion into a vein or body cavity.

FIG. 12 illustrates a cross-section view of a puncture needle assembly (600) engaged with a holder (500).

FIG. 13 illustrates a cross-section view of a puncture needle assembly (600) and holder (500) combination engaged with a device described herein.

1. A device (100) having a proximal end (20) and a distal end (40) for withdrawal, processing and storage of a liquid sample comprising:
a piston assembly (300) comprising a piston (302) dismountably attached at its proximal end (20) to an actuating rod (350),
a container (200) having a body (202) disposed with a cylindrical chamber (204) for slidable movement of the piston (302) therein, wherein the cylindrical chamber (204) is provided:
at the distal end (40) with a resealable septum (410) for co-operation with a puncture needle assembly (600) for withdrawal of the liquid sample, and
at the proximal end (20) with a stop member (206), which stop member (206) limits movement of the piston (300) in a proximal direction,
wherein an exterior of the container body (202) is dimensioned to fit inside a centrifuge rotor, and the container (200) is configured for breakability into two parts at a temperature of 0 deg C. or lower at a breakable zone (210).

2. The device (100) according to claim 1, wherein the container body (202) is weakened at the breakable zone (210).

3. The device (100) according to claim 1, wherein the actuating rod (350) is configured for non-returnable movement in a proximal (20) direction.

4. The device (100) according to claim 3, provided with a linear ratchet mechanism for non-returnable movement of the actuating rod (350) in the proximal (20) direction.

5. The device (100) according to claim 1, wherein the piston (302) and actuating rod (350) are configured such that the actuating rod (350) is dismountable from the piston (300) by application of an axial pulling force by the actuating rod (350) against the piston (302) engaged in the stop member (206).

6. The device (100) according to claim 1, wherein the piston (302) is dismountably attached to the actuating rod (350) by a latching joint, wherein the latching joint comprises:
a first part (320) on the piston (302) comprising one or more rounded protrusions; and
a second part (352) on the actuating rod (350) comprising one or more recesses or holes for retaining the rounded protrusion.

7. The device (100) according to claim 1, wherein the container body (202) at the proximal end (20) is bevelled, tapered, conical or rounded for insertion into a centrifuge rotor.

8. The device (100) according to claim 1, wherein the container body (202) is configured for centrifugation at at least 1000 g for at least 10 minutes.

9. The device (100) according to claim 1, wherein the device (100) is configured for to withstand freezing at a temperature of up to −200 deg C.

10. The device (100) according to claim 1, wherein the cylindrical chamber (204) is disposed with a composition comprising one or more substances having an antioxidant property and/or one or more substances having an anticoagulant property and/or tracing compounds.

11. The device (100) according to claim 1, wherein the distal end (40) of the body is configured to engage with a holder (500) disposed with a cylindrical passage (508) open at a proximal end (20) dimensioned to receive the distal end of the device (100), wherein the distal end (40) of the passage (508) terminates in a fitting (506) for connection to the puncture needle assembly (600).

12. The device (100) according to claim 1, further comprising an identifier, such as writable label, a writable space, a programmable or non-programmable RFID tag, an optically-readable 1- or 2-dimensional code.

13. The device (100) according to claim 1 for separation of erythrocytes from a blood sample, wherein after centrifugation of the blood sample in the container (200), the centrifuged sample in the container (200) is frozen, the container body (202) broken at the breakable zone (210), and the erythrocytes are retained in the proximal part of the cylindrical chamber (204),
wherein the piston (302) and actuating rod (350) of the device are configured such that the actuating rod (350) is dismountable from the piston (300) by application of an axial pulling force by the actuating rod (350) against the piston (302) engaged in the stop member (206).

14. A method for fractionating, and separating one or more fractions from a blood sample comprising:
obtaining a device (100) according to claim 1 containing the blood sample,
fractionating the sample by centrifugation,
freezing the device (100) after fractionation, and
breaking the container body (202) at the breakable zone (210),
wherein the one or more fractions are separated in one part of the container body (202) broken at the breakable zone (210).

15. The method of claim 14, wherein erythrocytes are separated from the blood sample.
